(19) United States
(12) Patent Application Publication
(10) Pub. No.: US 2022/0053637 A1
(43) Pub. Date: Feb. 17, 2022

(54) TEXTILE DEVICE CONFIGURED TO COOPERATE WITH AN ELECTRONIC DEVICE

(71) Applicant: BioSerenity, Paris (FR)

(72) Inventors: Alexandre ARISTIDES, Paris (FR); Pierre PROT, Paris (FR)

(73) Assignee: Bioserenity, Paris (FR)

(21) Appl. No.: 17/276,756

(22) PCT Filed: Sep. 20, 2019
(86) PCT No.: PCT/EP2019/075343

§ 371 Date: Mar. 16, 2021

(30) Foreign Application Priority Data
Sep. 21, 2018 (EP) 18306228.0

(51) Int. Cl. H05K 1/03; H05K 1/18; A41D 1/00

(52) U.S. Cl. CPC H05K 1/038; H05K 2201/10037; A41D 1/002; H05K 1/189

(57) ABSTRACT

The present invention relates to a textile device (1) configured to cooperate with an electronic device (2) comprising conductive path (21) having a path connection area (22). The textile device (1) comprises a first portion of textile (10) including a conductive zone (11) connected to a stretchable connection area (12) configured to be in contact with the path connection area (22) of the conductive path (21) so as to ensure an electrical connection between said textile device (1) and said electronic device (2). The stretchable connection area (12) comprises a conductive textile or a textile covered with a flexible conductive film. The textile device (1) also comprises a compartment, defined between the first portion of textile (10) and a second portion of textile (13), configured to maintain the electronic device (2) in a predefined position ensuring electrical connection between the stretchable connection area (12) and the path connection area (22).

### FIELD OF INVENTION

The present invention relates to the field of smart clothing and electronic circuit connection systems for connection with an electronic textile or smart clothing.

### BACKGROUND OF INVENTION

Smart clothing and textiles require the integration of electronics in media compatible with textile constraints. To withstand these constraints, the electronic equipment must be flexible, waterproof, and should have a limited thickness so as to avoid discomfort for the user. The electronics should also be able to withstand all the stresses experienced by the textile or clothing in its daily use (handling, washing, drying, folding . . . ).

Smart clothing often involves on-board electronic circuits. The electronic circuits comprise a set of electronic components often interconnected by means of a printed circuit. Electronic circuit technology has been developed for many years to become a mature technology, with reasonable cost, effective quality control, and an ability to integrate a high number of components compared to other technologies such as systems on chips. Another advantage in the use of electronic circuits is their interchangeability, for example, after the deterioration of a garment.

In this context, there is a necessity to develop a way to connect the electronic circuit to a textile. Several possibilities exist but still have many disadvantages. In particular, most existing connections involve a large thickness, protruding parts that catch on other clothes during washing, difficulties in positioning and insertion by the user or the presence of cables or exposed wires.

There is first of all the possibility of connecting the conductive threads of the textile to the tracks of the electronic circuit by welding in order to connect the conductive zone of the textile to the electronic circuit. This method makes it possible to limit both size and mass of the devices connected to the textile. However, the process of welding each component to each wire or conductive part of the textile is long and difficult to automate. In addition, this method tends to concentrate the stresses at the axis of the weld and increases the risk of breaking the conductive thread of the welded textile.

A method of installing clasps for connecting the electronic device and the textile is also known. The clasp is thus detachably connected, on one side to the textile, on the other to the electronic circuit. However, the connection is only made in one or more points, making the connection fragile in case of constraints on one end of the device and therefore providing an unstable connection. In addition, the rigidity inherent to such a connection method can provide discomfort for the user and further weaken the connection.

Another common solution consists in the use of snap fasteners, but as the number of connections increases, the area covered by the snap fasteners is too large for a good user comfort and fixing or removing the electronic module becomes difficult.

The present invention therefore aims to develop a new system comprising an electronic circuit configured for electrical and mechanical connection to a conductive area of an electronic textile and able to withstand the stresses undergone by the textile or garment in its daily use. The device according to the present invention is configured to be fixed to and detached from the textile without the implementation of complex operations. The device according to the present invention is further configured to be worn by the user of the textile without any discomfort.

### SUMMARY

The present invention relates to a textile device configured to cooperate with an electronic device comprising a conductive path having a path connection area, the textile device comprising:
a first portion of textile including a conductive zone connected to a stretchable connection area configured to be in contact with the path connection area of the electronic device so as to ensure an electrical connection between said textile device and said electronic device, wherein the stretchable connection area comprises a conductive textile or a textile covered with a flexible conductive film; and
a compartment, defined between the first portion of textile and a second portion of textile, configured to maintain the electronic device in a predefined position, relative to the textile device, ensuring the electrical connection between the stretchable connection area and the path connection area.

Within the meaning of the invention, a conductive textile is a textile whose electrical conductivity results from the textile fibers themselves, which may be yarns made of a conductive material or non-conductive yarns covered with a conductive material, either individually by covering the external surface of each yarn or collectively by printing a surface of the textile with a conductive ink.

Such a structure of the textile device advantageously provides a compartment configured to receive an electronic device and create an electrical connection between the textile device and the electronic device able to withstand the stresses undergone by the textile. More precisely, thanks to the mechanical properties of the stretchable connection area, which comprises a conductive textile or a textile covered with a flexible conductive film, the risk of tearing the textile device in the vicinity of the connection area is significantly reduced, which improves the mechanical resistance and the durability of the textile device.

The first and/or the second portions of textile of the textile device may be made of a material obtained by a thread assembly. The use of a textile comprising conductive zones makes it possible to produce smart clothes which are compact and easy to wear because all conductive tracks are integrated into the textile itself.

In one embodiment, the first portion of textile is made of an elastic textile material.

In one embodiment, the conductive zone of the first portion of textile is obtained by weaving or knitting conductive yarns made of a conductive material, or comprising conductive material. According to one embodiment, the conductive material is silver.

In one embodiment, the conductive zone of the first portion of textile is obtained by weaving or knitting conductive yarns covered with a conductive material. In one preferred embodiment, the conductive zone of the first portion of textile is obtained by weaving or knitting yarns covered with a conductive metal such as silver.

In one embodiment, the conductive zone of the first portion of textile is obtained by printing with a conductive ink or a conductive paint on the first portion of textile. Said conductive ink or conductive paint is typically loaded with an electrically conductive material, having properties of flexibility allowing the conductive ink or conductive paint to be deposited on flexible surfaces.

In one embodiment, the conductive zone is obtained by deposition of a film with electrically conductive properties on the first portion of textile. In one embodiment, the conductive zone comprises at least one layer of electrically conductive ink coupled to a surface of the textile by means of at least one thermo-adhesive layer made of thermo-adhesive material and at least one elastic membrane placed between the first textile surface and the conductive path. Said film on the conductive ink may comprise one conductive metal or a combination of conductive metals such as silver, coper, other metals or carbon.

According to one embodiment, the first textile portion comprises more than one conductive zone each connected to one stretchable connection area. In one embodiment, the electronic device comprises more than one conductive path each connected to one path connection area. In one embodiment, the number of conductive zones on the first textile portion is equal to the number of conductive paths on the electronic device.

In one embodiment, the conductive zone is configured to be put in electrical contact with at least one conductive path of the electronic device in order to establish an electrical connection between the textile device and the electronic device. According to one embodiment, the connection area is located at one end of the conductive zone. According to an alternative embodiment, multiple connections areas are distributed along the conductive zone so as to establish for each one an electrical connection with at least one conductive path of the electronic device.

According to one embodiment, the electronic device configured to cooperate with the textile device is an electronic circuit, notably a printed electronic circuit including edges. According to one embodiment, the path connection area is positioned in vicinity or on one of the edges of the electronic device. According to one embodiment, the path connection area is a conductive area having a predefined surface. Said predefined surface may be enlarged so to present a larger contact zone to the stretchable connection area in order to improve the quality of the electrical connection.

According to one embodiment, the second portion of textile is mechanically connected to the first portion of textile in order to define the compartment. According to one embodiment, the second portion of textile is sewn to the first portion of textile. In one alternative embodiment, the second portion of textile is mechanically connected to the first portion of textile by thermal adhesive bonding.

The second portion of textile may be made of a material obtained by a thread assembly. According to one embodiment, the second portion of textile is made of an elastic material.

According to one embodiment, the compartment, defined between the first portion of textile and the second portion of textile, is made of an elastic material or elastic fibers.

According to one embodiment, the compartment is a pocket obtained by sewing the second portion of textile onto the first portion of textile. According to this embodiment, the pocket is configured to allow the introduction of the electronic device by sliding it through the opening of the pocket. Then, the electronic device may be easily slid in and out of the pocket compartment.

According to one embodiment, the compartment has dimensions configured to maintain the electronic device in a predefined position, said predefined position allowing to position the stretchable connection area of the textile facing the respective path connection area of the electronic device so as to ensure both physical contact and electrical connection between the stretchable connection area and the path connection area when the electronic device is positioned inside the compartment of the textile device. The length and width dimensions of the compartment may range approximately from 1 cm to 10 cm, so as to be adapted to an electronic device having length and width dimensions ranging approximately from 1 cm to 10 cm and height comprised between approximately between 0.5 mm and 30 mm. The elasticity of the second portion of textile advantageously applies a force to the electronic device when it is placed in the pocket, thus contributing to maintaining the electronic device in the predefined position.

The first portion of textile and/or the second portion of textile defining the compartment may comprise at least one blocking means to block the electronic device inside the compartment and maintain the electronic device in the predefined position. Said at least one blocking means may be a pressure button or a Velcro strap.

According to one embodiment, the stretchable connection area comprises a conductive textile obtained by weaving or knitting conductive yarns made of a conductive material or comprising a conductive material such as for example silver.

According to one embodiment, the stretchable connection area comprises a conductive textile obtained by weaving or knitting conductive yarns covered with a conductive material, preferably by weaving or knitting yarns covered with a conductive metal such as silver.

According to one embodiment, the stretchable connection area comprises a conductive textile obtained by printing a portion of textile with a conductive ink or conductive paint. The conductive ink or conductive paint is loaded with an electrically conductive material, having properties of flexibility allowing the conductive ink or conductive paint to be deposited on flexible surfaces.

According to one embodiment, the stretchable connection area comprises a textile covered with a flexible conductive film. In one embodiment, the conductive zone comprises at least one layer of electrically conductive ink coupled to a surface of the textile by at least one thermo-adhesive layer made of thermo-adhesive material, at least one elastic membrane placed between the first textile surface and the conductive path. Said film or conductive ink may comprise one conductive metal or a combination of conductive metals such as silver, coper, other metals or carbon.

According to one embodiment, the stretchable connection area is in the first portion of textile and/or the second portion of textile. One stretchable connection area may comprise a first partial stretchable connection on the first portion of textile and a second partial stretchable connection on the second portion of textile where the first and second partial stretchable connections are mechanically connected when the second portion of textile is mechanically connected to the first portion of textile forming the compartment. In one alternative embodiment, the stretchable connection area is entirely comprised in the first portion of textile like the conductive zone to which is connected. In this embodiment, the stretchable connection area is positioned in the first portion of textile so as to be in the vicinity or in contact with one of the edges of the compartment that is formed when the second portion of textile is mechanically connected to the first portion of textile. In one embodiment, the stretchable connection area is entirely comprised in the second portion of textile and the stretchable connection area is electrically and mechanically connected with the conductive zone on the first portion of textile when the second portion of textile is mechanically fixed to the first portion of textile so as to define the compartment. According to one embodiment, the stretchable connection area is positioned at the bottom of the compartment.

The use of a stretchable connection area instead of a rigid body inserted in the textile has the advantage of providing a structure that is more robust to stresses applied to the textile, so as to cause less inelastic deformations of the textile itself while preserving the electrical connection between the electronic device and the textile. Indeed, when a stress is applied to the textile, the connection areas of the electronic device and the textile device, which are not rigidly fixed to each other since one of the interacting surfaces is a conductive textile, are allowed to undergo a relative displacement while maintaining an electrical connection. On the contrary, a rigid body inserted through or on the textile which is rigidly fixed to the electronic device does not allow a relative displacement of the connection areas and, in case of stress applied to the textile, the rigid body transfers the stress directly to the textile structure, thus causing its degradation.

In one embodiment, edges of the compartment where the first portion of textile is mechanically connected to the second portion of textile are straight and do not comprise any protrusion or notch.

According to one embodiment, the electronic device and the compartment includes edges having complementary features in relief in the vicinity of the connection areas so that the stretchable connection area is in contact with the path connection area when the complementary features in relief are mutually engaged.

According to one embodiment, the compartment comprises at least one first feature in relief in the vicinity of the stretchable connection area, the or each first feature in relief being complementary to a second feature in relief comprised by the electronic device in the vicinity of the path connection area.

According to one embodiment, the or each feature in relief of the compartment in the vicinity of a stretchable connection area is formed at the bottom of the compartment, so that the complementary features in relief of the electronic device and the compartment become mutually engaged when the electronic device abuts against the bottom of the compartment. The complementary features in relief may be notches and protrusions.

According to one embodiment, the first features in relief are notches and the second features in relief are protrusions, or inversely.

According to one embodiment, the compartment comprises at one of its edges at least one notch and at least one protrusion in proximity of the at least one stretchable connection area, each pair of notch and protrusion being configured to cooperate with a complementary pair of protrusions and notch of the electronic device.

In these embodiments, the shape of the notch and/or protrusion is complementary to the shape of the protrusion and/or notch to ensure an optimal mechanical contact and electrical connection between the conductive zone of the textile and the electronic device. The maximum depth of the notch may range from 0.001 mm to 10 mm, whereas the protrusion may have a height ranging from 0.001 mm to 10 mm.

In one embodiment, the textile device comprises a plurality of shielding zones separating from one another the one or more conductive zones, in order to reduce the electronic noise.

The present invention further relates to electronic textile comprising a textile device, according to any one of the embodiments described hereabove, and an electronic device.

According to one embodiment, the electronic device comprises a path connection area and at least one conductive path extending from said path connection area.

In one embodiment, the electronic device comprises an electronic circuit. In one embodiment, said electronic circuit is a flexible electronic circuit, in particular a flexible printed circuit. A flexible printed circuit, thanks to its flexibility, allows the manufacturing of garments which are more comfortable than garments comprising a rigid electronic circuit.

Flexible electronic circuit technology is well known to those skilled in the art. Flexible electronic circuit usually consists in using a high-performance plastic substrate, such as polyimide. A flexible electronic circuit is an electrical and mechanical support of electronic components. It is made on a flexible support of the polyamide, polyetheretherketone (PEEK), polyester (PE) or other type. This flexible support makes it possible to fold the electronic device or to deform it without breaking the flexible electronic circuit. This flexible support can be reinforced by semi-rigid encapsulation in a resin, which ensures a minimum rigidity of the circuit while maintaining sufficient ability to be bent. The electronic device can also be obtained on a rigid support, such as a rigid support of epoxy resin type reinforced with glass fibers (FR-4).

According to one embodiment, the electronic circuit is put in electrical contact with the stretchable connection area when the electronic device is introduced in the compartment.

According to one embodiment wherein the conductive paths of the textile device are connected to electrical sensors, the electronic device is capable of recording or analyzing a signal from the textile device. The electronic circuit may also be able to respond to a signal or transmit a signal. Signal transmission may be done through wires or wirelessly, for example using optic, induction or radio communication or the like.

In one embodiment, the components performing electronic functions are supported by the printed circuit board (PCB) of the electronic circuit. In one embodiment, these electronic components are soldered to the printed circuit of the electronic circuit. In one embodiment, the electronic circuit comprises layers of conductive materials, preferably copper coated with nickel and/or gold to prevent oxidation, so as to obtain conductive tracks. These tracks electrically connect different areas of the electronic circuit, either component-component or component to at least one connection track which is an entry point of the flexible electronic circuit. In one embodiment not shown, the electronic circuit is covered with a layer of varnish that protects the tracks from oxidation and possible short circuits.

In one embodiment, the electronic device is equipped with rigid reinforcements for stiffening certain areas under the electronic components whose welding process is incompatible with the flexible electronic circuits. In this embodiment, the electronic circuit is a succession of rigid zones articulated together by flexible zones.

According to one embodiment, the electronic device further comprises an oximeter, comprising one or more light-emitting diodes (LEDs) and one or more photodiodes. In one embodiment, the first portion of textile comprises an opening, the oximeter and the opening being configured to face each other when the electronic device is positioned in the compartment. The oximeter may be disposed on the side of the electronic device which, when inserted in the compartment, faces the first portion of textile while the opening on the first portion of textile may be configured so that, when the electronic device is positioned in the compartment, the oximeter faces the opening.

The electronic device may be further equipped with a battery, either rigid or flexible, which may be applied on the PCB or directly integrated within the resin of the module.

In one embodiment, the battery is mechanically and electrically connected to the PCB with at least one mechanical and electrical connection means, notably a clip. In this embodiment, a first battery is easily removable from the PCB to be, for example, replaced by a second already charged battery, during a period of recharge of the first battery.

In one alternative embodiment, the battery is not removably fixed to the PCB and is configured to be recharged using a connector or by wireless power transfer, i.e. by transmission of electrical energy without wires as a physical link. In one example, a telephone device of the user may be used to recharge by inductive charging the battery of the textile device. In one embodiment, the textile device implements a low power inductive transfer delivering power below 5 W using inductive coupling between two planar coils, which operate with the Qi standard in order to perform wireless power transfer between the battery and an external electronic device.

According to one embodiment, the textile device comprises a second pocket having dimensions adapted to take and hold the external electric device and allowing the positioning of the external electric device in proximity to the battery so as to perform wireless power transfer. In one example, a third portion of textile may be sewn onto the first portion of textile or onto the second portion of textile so as to define a second pocket which overlaps with the compartment.

The present invention further relates to an electronic garment comprising an electronic textile according to any one of the embodiments described hereabove. According to one embodiment, the first portion of textile is part of the electronic garment. Said electronic garment may be a shirt, a t-shirt, a pair of trousers, a hat, a band, a sock, a glove, a panty, a tight, an armband, a bandeau, an ankle or shoulder pad, and the like.

According to one embodiment, the electronic device comprises an oximeter and the first portion of textile comprises an opening, said oximeter and said opening being configured to be facing each other when the electronic device is positioned in the compartment, so that the oximeter is positioned in proximity or in contact with the skin of a subject when the electronic garment is worn by the subject.

This advantageously allows a smart garment to perform measurements continuously over long periods of time (such as several days). When the patient changes clothes, he or she can then remove the electronic device from an old garment and put it on a new garment, without the help of a technician. The patient is therefore autonomous and can perform this kind of measurement without modifying his/her daily activity.

### Definitions

In the present invention, the following terms have the following meanings:
“Textile” refers to a material or product manufactured by textile fibers. The textile may, for example, be obtained by assembling yarns, fibers and/or filaments by any method such as, for example, weaving, braiding, or knitting, or non-woven, or any other known method in the art.
“Smart clothing”: refers to any textile capable of being worn by a subject comprising at least one conductive zone configured to transmit or receive an electrical signal.
“Printed circuit board”: refers to a conductive wiring system wherein the conductive material is printed on the board and different electrical components can be bonded to the conductive wiring system, further wherein, each set of different electrical components can achieve a different purpose.

### DESCRIPTION OF THE DRAWINGS

Features and advantages of the invention will become apparent from the following description of embodiments of a textile device, an electronic textile and an electronic garment according to the invention, this description being given merely by way of example and with reference to the appended drawings in which:

FIG. 1 is a representation of a textile device according to a first embodiment of the present invention, where the electronic device is not inserted in the first portion of textile, and a portion of the electronic device.

FIG. 2 is a representation the textile device according to the first embodiment of the present invention, where the electronic device is inserted inside the compartment.

FIG. 3 is a zoomed-out representation of a textile device according to a second embodiment of the present invention.

FIG. 4 is a schematic representation of an electronic garment according to the invention, comprising the textile device of FIG. 2 or 3 as part of the garment.

### ILLUSTRATIVE EMBODIMENTS OF THE INVENTION

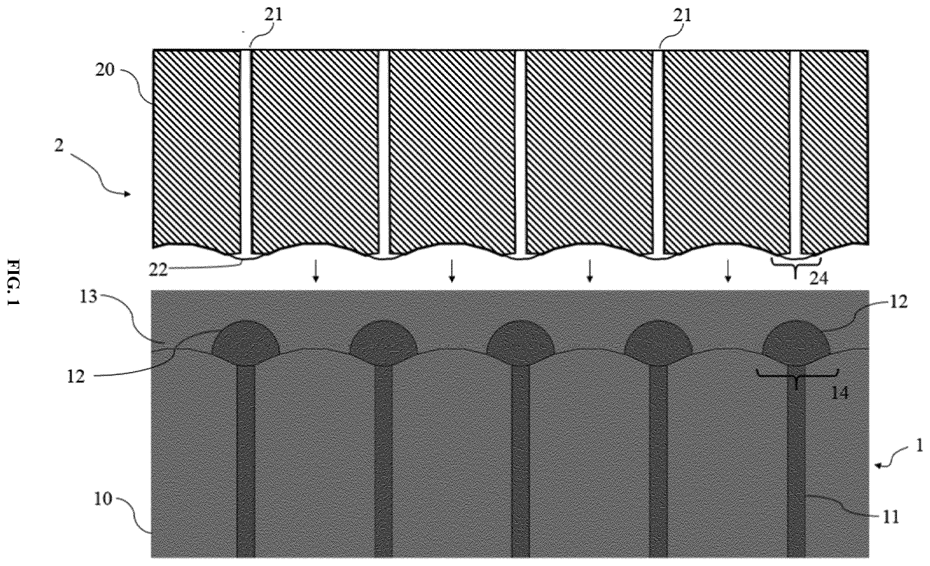

As shown in FIG. 1, the textile device 1 according to the first embodiment comprises a first textile portion 10. Said first textile portion 10 comprises more than one conductive zones 11 that can be obtained by the insertion in the textile of conductive yarns by weaving or knitting. FIG. 1 also shows a portion of an electronic device 2 comprising multiple conductive paths 21, each having one path connection area 22 positioned at the edge of the electronic device 2.

In this first embodiment, each of the conductive zones 11 comprises one stretchable connection area 12 configured to be in contact with the path connection area 22 of the conductive path 21 so as to ensure an electrical connection between the textile device 1 and the electronic device 2, wherein the stretchable connection areas 12 comprise a conductive textile or a textile covered with a flexible conductive film.

According to the first embodiment, the textile device 1 further comprises a second portion of textile 13 mechanically connected to the first portion of textile 10 so as to define a compartment into which the electronic device 2 can be slide, the sliding direction being designed by the arrows in FIG. 1.

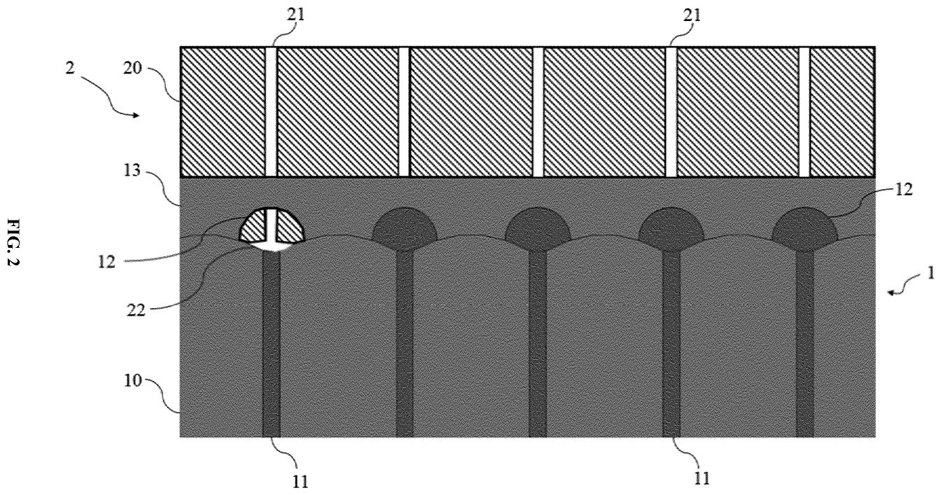

As illustrated in FIG. 2, the compartment is configured so as to maintain the electronic device 2 in a predefined position ensuring the physical contact and therefore the electronic connection between the stretchable connection areas 12 of the portion of textile 10 and each of the facing path connection area 22 of the electronic device 2. This feature allows to keep the electronic device operatively connected to the textile device 1 when the garment comprising the first textile portion 10 is wore by a subject in activity.

In this first embodiment, the electronic device 2 and the compartment include edges having complementary features in relief (14, 24) respectively in the vicinity of the stretchable connection area 12 and path connection area 22, so that the stretchable connection area 12 is in contact with the path connection area 22 when the complementary features in relief are mutually engaged (14, 24).

In FIG. 1, the complementary features in relief in proximity of each stretchable connection area 12 located at the bottom of the compartment are notches 14. The electronic device 2 comprises along one of its edges as many complementary features in relief, in the shape of protrusions 24, as the number of notches 14 on the compartment. The protrusions 24 on the electronic device 2 have a flat semi-circular shape and dimensions adapted to fit into the notches of the compartment 13.

According to this first embodiment, the stretchable connection areas 12 is in the first portion of textile 10 (not shown) and in the second portion of textile 13. The stretchable connection areas 12 in the second portion of textile 13 has an enlarged conductive area with a semi-circular shape.

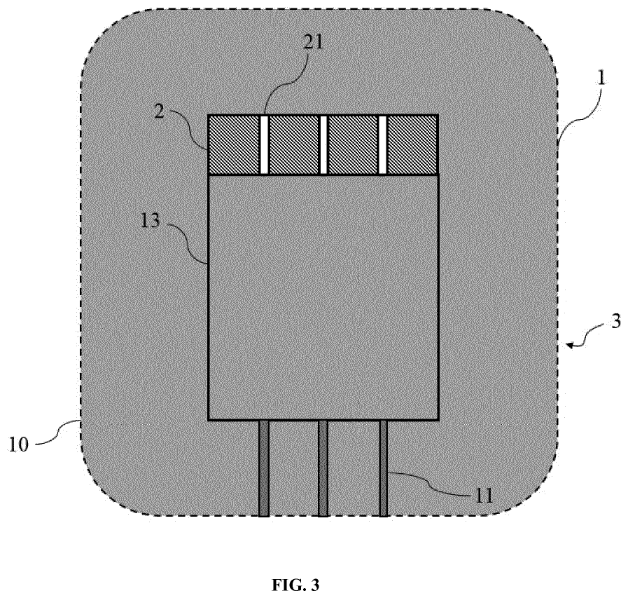

In the second embodiment shown in FIG. 3, the compartment is a pocket obtained by mechanical coupling of the first portion of textile 10 and the second portion of textile 13. Therefore, the electronic device 2 may be easily slide in and out of the pocket. In this second embodiment, where no additional blocking means is used, the compartment is made of elastic material or elastic fibers so as to exert a constant blocking force on the electronic device 2 and prevent it from sliding out of the compartment 13.

In this second embodiment, the edges of the pocket mechanically connected to the first portion of textile 10 do not have notches and the contact between the conductive zones 11 of the first textile portion 10 and the conductive paths 21 of the electronic device 2 is made at the bottom of the pocket.

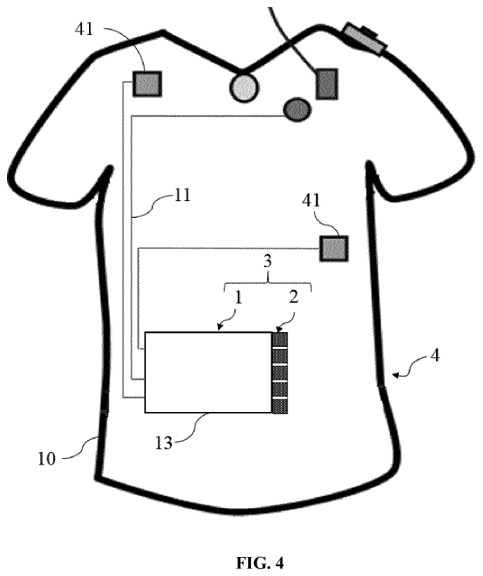

FIG. 4 shows an electronic garment 4 comprising a textile device 1 according to the second embodiment shown in FIG. 2 or FIG. 3.

In this embodiment, the textile device 1 is part of the garment 4 and the conductive paths 11 of the textile device 1 are connected to multiple electrical sensors 41. These sensors may be electrophysiological sensors such as electrodes for electrocardiographic signal recording, for electromyographic signal recording and the like.

### REFERENCES

1—Textile device;
2—Electronic device;
3—Electronic textile;
10—First portion of textile;
11—Conductive zone of the first portion of textile;
12—First connection area;
13—Compartment;
21—Conductive path;
22—Path connection area;
4—Garment;
41—Sensor.

1. A textile device configured to cooperate with an electronic device comprising a conductive path having a path connection area, the textile device comprising:
a first portion of textile including a conductive zone connected to a stretchable connection area configured to be in contact with the path connection area so as to ensure an electrical connection between the textile device and the electronic device, wherein the stretchable connection area comprises a conductive textile or a textile covered with a flexible conductive film; and
a compartment, defined between the first portion of textile and a second portion of textile, configured to maintain the electronic device in a predefined position, relative to the textile device, ensuring electrical connection between the stretchable connection area and the path connection area,
wherein the electronic device and the compartment include edges having complementary features in relief in a vicinity of the connection areas so that the stretchable connection area is in contact with the path connection area when the complementary features in relief are mutually engaged.

2. The textile device according to claim 1, wherein the stretchable connection area is in the first portion of textile and/or the second portion of textile.

3. (canceled)

4. The textile device according to claim 1, wherein the stretchable connection area comprises a conductive textile obtained by weaving or knitting conductive yarns made of a conductive material or covered with a conductive material, preferably by weaving or knitting yarns covered with a conductive metal such as silver.

5. The textile device according to claim 1, wherein the stretchable connection area comprises a conductive textile obtained by printing the portion of textile with a conductive ink.

6. The textile device according to claim 1, wherein the second portion of textile is made of an elastic material.

7. The textile device according to claim 1, wherein the second portion of textile is sewn to the first portion of textile.

8. The textile device according to claim 1, wherein the compartment is a pocket obtained by sewing the second portion of textile onto the first portion of textile.

9. The textile device according to claim 1, wherein the conductive zone of the textile is obtained by weaving or knitting conductive yarns made of a conductive material or covered with a conductive material, preferably by weaving or knitting yarns covered with a conductive metal such as silver.

10. The textile device according to claim 1, wherein the conductive zone of the textile is obtained by deposition of a conductive film.

11. The textile device according to claim 1, wherein the conductive zone of the textile is obtained by printing with a conductive ink.

12. An electronic textile comprising:
a textile device according to claim 1, and
an electronic device comprising a path connection area and at least one conductive path extending from the path connection area.

13. The electronic textile according to claim 11, wherein the electronic device comprises a flexible printed circuit board and a battery fixed on the flexible printed circuit board.

14. An electronic garment comprising an electronic textile according to claim 11, wherein the first portion of textile is part of the electronic garment.

15. The electronic garment according to claim 14, wherein the electronic device comprises an oximeter and the first portion of textile comprises an opening, the oximeter and the opening being configured to be facing each other when the electronic device is positioned in the compartment.
